(19) United States
(12) Patent Application Publication
(10) Pub. No.: US 2009/0186864 A1
(43) Pub. Date: Jul. 23, 2009

(54) RUTHENIUM (II) COMPOUNDS

(76) Inventors: Abraha Habtemariam, Edinburgh (GB); Tijana Bugarcic, Edinburgh (GB); Peter John Sadler, Edinburgh (GB)

Correspondence Address: CURATOLO SIDOTI CO., LPA, 24500 CENTER RIDGE ROAD, SUITE 280, CLEVELAND, OH, 44145, US

(21) Appl. No.: 12/301,373

(22) PCT Filed: May 21, 2007
(86) PCT No.: PCT/GB2007/001874

§ 371 Date: Jan. 21, 2009

(30) Foreign Application Priority Data
May 19, 2006 (GB) 0610062.2

(51) Int. Cl. A61K 31/555; C07F 15/00; A61P 35/00

(52) U.S. Cl. 514/185; 546/4

(57) ABSTRACT

A ruthenium (II) compound of formula (I):

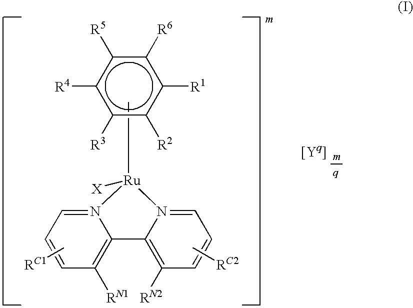

or a solvate form thereof for use in a method of therapy, wherein:
R¹, R², R³, R⁴, R⁵ and R⁶ are independently selected from H, C₁₋₇ alkyl, C₅₋₂₀ aryl, C₃₋₂₀ heterocyclyl, halo, ester, amido, acyl, sulfo, sulfonamido, ether, thioether, azo and amino, or R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other carbocyclic or heterocyclic rings;
X is halo or a neutral or negatively charged O, N— or S— donor ligand;
Y is a counterion;
m is −1, 0, 1 or 2;
q is 1, 2 or 3;
R^{C1} and R^{C2} independently represent one or more optional substituents selected from hydroxy, C₁₋₇ alkoxy, C₅₋₂₀ aryloxy, C₁₋₇ alkyl, carboxy, C₁₋₇ alkyl ester and C₅₋₂₀ aryl ester;
R^{N1} and R^{N2} are independently selected from hydroxy, C₁₋₇ alkoxy, C₅₋₂₀ aryloxy, C₁₋₇ alkyl, carboxy, C₁₋₇ alkyl ester and C₅₋₂₀ aryl ester;
or R^{N1} and R^{N2} together with the pyridine rings to which they are bound form an tricyclic heteraromatic moiety, where the ring formed by R^{N1} and R^{N2} together may be optionally substituted by one or more substituents represented by R^{C3} selected from: hydroxy, C₁₋₇ alkoxy, C₅₋₂₀ aryloxy, C₁₋₇ alkyl, carboxy, C₁₋₇ alkyl ester and C₅₋₂₀ aryl ester.

This invention relates to ruthenium (11) compounds, to their use in medicine, particularly for the treatment and/or prevention of cancer, and to a process for their preparation.

WO 01/30790, WO 02/02572, WO 2004/005304 and WO 2004/096819 disclose ruthenium (II) compounds for use in the treatment of cancer. These compounds can be described as half-sandwich compounds, having an arene ring bound to the ruthenium, as well as other non-arene ligands. The compounds exemplified in these applications have as one of the ligands a halo atom. It is thought that the hydrolysis of the halo atom activates the complexes and allows them to bind to DNA. More recently it has been found that complexes containing ligands that have longer hydrolysis times still exhibit anti-tumour activity (Sadler et al, Proc. Natl. Acad. Sci. USA, 2005, 102, 18269).

The following complex was disclosed in a poster at the 1^{st} European Conference on Chemistry for Life Sciences, Rimini, Italy, Oct. 4-8 2005 (Habetemariam, A., et al., Organometallic Ruthenium Arene Anticancer Complexes: Structure-Activity Relationships):

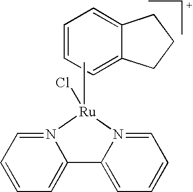

Its activity (IC₅₀) in inhibiting the growth of A2780 human ovarian cancer cells (as measured by the method of Example 7) was quoted as being >100 μM, i.e. essentially inactive.

The present inventors have discovered that substituted analogues of the above compound surprisingly show anti-tumour activity.

According to a first aspect of the present invention there is provided a ruthenium (II) compound of formula (I):

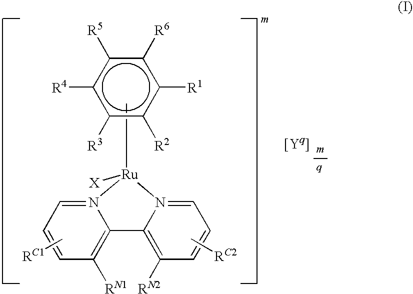

or a solvate form thereof for use in a method of therapy, wherein:
R¹, R², R³, R⁴, R⁵ and R⁶ are independently selected from H, C₁₋₇alkyl, C₅₋₂₀ aryl, C₃₋₂₀ heterocyclyl, halo, ester, amido, acyl, sulfo, sulfonamido, ether, thioether, azo, amino, or R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other carbocyclic or heterocyclic rings;
X is halo or a neutral or negatively charged O, N— or S— donor ligand;
Y is a counterion;
m is −1, 0, 1 or 2;
q is 1, 2 or 3;
R^{C1} and R^{C2} independently represent one or more optional substituents selected from hydroxy, C₁₋₇ alkoxy, C₅₋₂₀ aryloxy, C₁₋₇ alkyl, carboxy, C₁₋₇ alkyl ester and C₅₋₂₀ aryl ester;
R^{N1} and R^{N2} are independently selected from hydroxy, C₁₋₇ alkoxy, C₅₋₂₀ aryloxy, C₁₋₇ alkyl, carboxy, C₁₋₇ alkyl ester and C₅₋₂₀ aryl ester;
or R^{N1} and R^{N2} together with the pyridine rings to which they are bound form an tricyclic heteraromatic moiety, where the ring formed by R^{N1} and R^{N2} together may be optionally substituted by one or more substituents represented by R^{C3} selected from: hydroxy, C₁₋₇ alkoxy, C₅₋₂₀ aryloxy, C₁₋₇ alkyl, carboxy, C₁₋₇ alkyl ester and C₅₋₂₀ aryl ester.

The compound of structure:

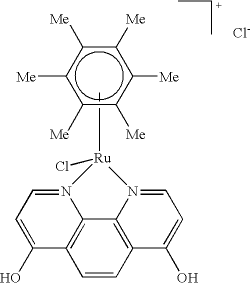

is disclosed in JP 2004-217632 and Himeda, Y., et al., Organometallics, 2004, 23, 1480-1483 as a catalyst for the hydrogenation of bicarbonate.

The following compounds:

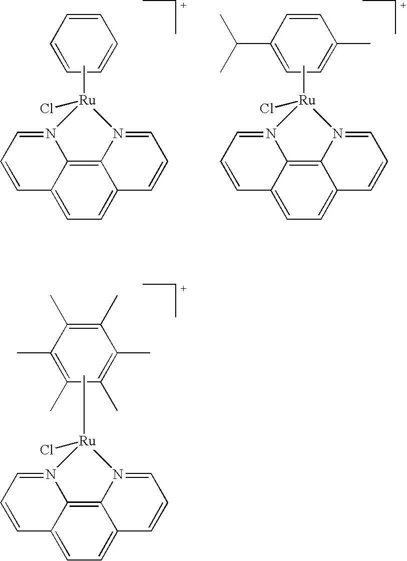

and their corresponding aqua complexes are disclosed in Stephicka, P, et al., Inorgnanica Chimica Acta, 359, 2369-2374 (2006) as catalysts for transfer hydrogenation. Their synthesis was reported in Canivet, J., et al., J. Organomet. Chem., 690, 3202-3211 (2005).

The following compounds:

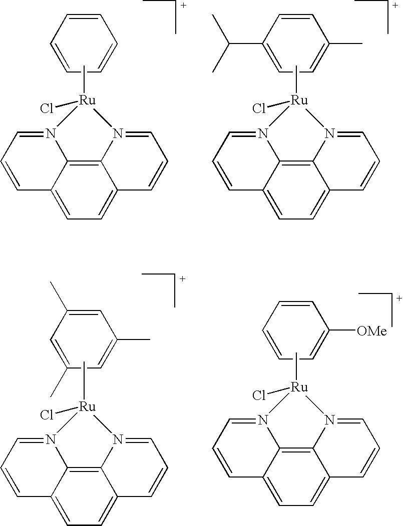

were described in Robertson, D., et al., J. Organomet. Chem., 202, 309-318 (1980).

The following compound:

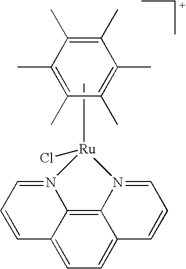

was described in JP 2004-224715 as a catalyst.

A second aspect of the present invention provides a pharmaceutical composition comprising a ruthenium (II) compound as described in the first aspect and a pharmaceutically acceptable carrier or diluent.

A third aspect of the invention provides the use of a compound as described in the first aspect in the preparation of a medicament for the treatment of cancer. This aspect also provides a compound as described in the first aspect for use in a method of treating cancer.

A fourth aspect of the invention provides a method of treatment of a subject suffering from cancer, comprising administering to such a subject a therapeutically-effective amount of a compound as described in the second aspect, preferably in the form of a pharmaceutical composition.

A fifth aspect of the invention provides a ruthenium (II) compound of formula (I):

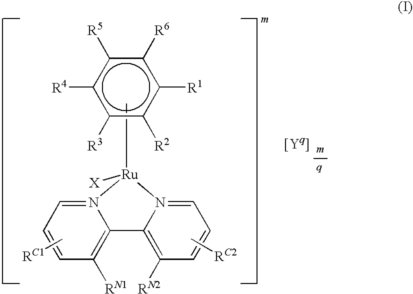

or a solvate form thereof, wherein:
R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other carbocyclic or heterocyclic rings; or
R¹ is C₅₋₂₀ aryl, and R² is selected from H, C₁₋₇ alkyl, C₅₋₂₀ aryl, C₃₋₅₀ heterocyclyl, halo, ester, amido, acyl, sulfo, sulfonamido, ether, thioether, azo and amino;
R³, R⁴, R⁵ and R⁶ are independently selected from H, C₁₋₇ alkyl, C₅₋₂₀ aryl, C₃₋₂₀ heterocyclyl, halo, ester, amido, acyl, sulfo, sulfonamido, ether, thioether, azo, amino, or
X is halo or a neutral or negatively charged O, N— or S— donor ligand;
Y is a counterion;
m is −1, 0, 1 or 2;
q is 1, 2 or 3;
R^{C1} and R^{C2} independently represent one or more optional substituents selected from hydroxy, C₁₋₇ alkoxy, C₅₋₂₀ aryloxy, C₁₋₇ alkyl, carboxy, C₁₋₇ alkyl ester and C₅₋₂₀ aryl ester;

R^{N1} and R^{N2} are independently selected from hydroxy, C₁₋₇ alkoxy, C₅₋₂₀ aryloxy, C₁₋₇ alkyl, carboxy, C₁₋₇ alkyl ester and C₅₋₂₀ aryl ester; or R^{N1} and R^{N2} together with the pyridine rings to which they are bound form an tricyclic heteraromatic moiety, where the ring formed by R^{N1} and R^{N2} together may be optionally substituted by one or more substituents represented by R^{C3} selected from: hydroxy, C₁₋₇ alkoxy, C₅₋₂₀ aryloxy, C₁₋₇ alkyl, carboxy, C₁₋₇ alkyl ester and C₅₋₂₀ aryl ester.

The compounds of this aspect of the invention may be characterised as having a fused arene system, or an arene system which comprises a benzene ring bearing at least one aromatic substituent.

A sixth aspect of the present invention provides a ruthenium (II) compound of formula (I):

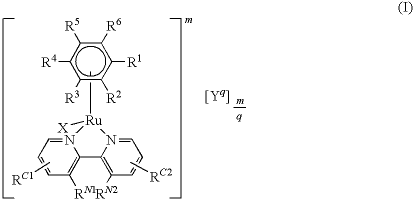

or a solvate form thereof, wherein:
R¹, R², R³, R⁴, R⁵ and R⁶ are independently selected from H, C₁₋₇ alkyl, C₃₋₂₀ heterocyclyl, halo, ester, amido, acyl, sulfo, sulfonamido, ether, thioether, azo and amino;
X is halo or a neutral or negatively charged O, N— or S— donor ligand;
Y is a counterion;
m is −1, 0, 1 or 2;
q is 1, 2 or 3;
R^{C1} and R^{C2} independently represent one or more optional substituents selected from hydroxy, C₁₋₇ alkoxy, C₅₋₂₀ aryloxy, C₁₋₇ alkyl, carboxy, C₁₋₇alkyl ester and C₅₋₂₀ aryl ester;

R^{N1} and R^{N2} are independently selected from hydroxy, C₁₋₇ alkoxy, C₅₋₂₀ aryloxy, C₁₋₇ alkyl, carboxy, C₁₋₇ alkyl ester and C₅₋₂₀ aryl ester.

The compounds of this aspect may be characterised as having an arene system that is an optionally substituted benzene ring, where the substitutents are not aromatic, and where the main ligand does not include phenanthroline or derivatives thereof.

### DEFINITIONS

N-donor ligands: N-donor ligands are ligands which bind to a metal atom via a nitrogen atom. They are well known in the art and include: nitrile ligands (N≡C—R); azo ligands (N≡N—R); aromatic N-donor ligands; amine ligands (NR^{N3}R^{N4}R^{N5}); azide (N₃⁻); cyanide (N≡C⁻); isothiocyanate (NCS⁻).

In both nitrile and azo ligands R may be selected from C₁₋₇ alkyl and C₅₋₂₀ aryl.

Aromatic N-donor ligands include optionally substituted pyridine, pyridazine, pyrimidine, purine and pyrazine. The optional substituents may be selected from cyano, halo and C₁₋₇ alkyl.

R^{N3}, R^{N4} and R^{N54} may be independently selected from H and C₁₋₇ alkyl.

S-donor ligands: S-donor ligands are ligands which bind to a metal atom via a sulphur atom. They are well known in the art and include: thiosulfate (S₂O₃²⁻); isothiocyanate (NCS⁻); thiocyanate (CNS⁻); sulfoxide ligands (R^{S1}R^{S2}SO); thioether ligands (R^{S1}R^{S2}S); thiolate ligands (R^{S1}S⁻); sulfinate ligands (R^{S1}SO₂⁻); and sulfenate ligands (R^{S1}SO⁻), wherein R^{S1} and R^{S2} are independently selected from C₁₋₇ alkyl and C₅₋₂₀ aryl, which groups may be optionally substituted.

O-donor ligands: O-donor ligands are ligands which bind to a metal atom via an oxygen atom. They are well known in the art and include: water (H₂O), carbonate (C₃); carboxylate ligands (R^{C}CO₂⁻); nitrate (NO₃⁻); sulfate (SO₄²⁻) and sulphonate (R^{S1}O₃⁻), wherein R^{C} is selected from C₁₋₇ alkyl and C₅₋₂₀ aryl and R^{S1} is as defined above.

C₁₋₇ Alkyl: The term “C₁₋₇ alkyl”, as used herein, pertains to a monovalent moiety obtained by removing a hydrogen atom from a carbon atom of a hydrocarbon compound having from 1 to 7 carbon atoms, which may be aliphatic or alicyclic, and which may be saturated or unsaturated (e.g., partially unsaturated, fully unsaturated). Thus, the term “alkyl” includes the sub-classes alkenyl, alkynyl, cycloalkyl, cycloalkyenyl, cylcoalkynyl, etc., discussed below.

Examples of saturated C₁₋₇ alkyl groups include, but are not limited to, methyl (C₁), ethyl (C₂), propyl (C₃), butyl (C₄), pentyl (C₅), hexyl (C₆) and heptyl (C₇).

Examples of saturated linear C₁₋₇ alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of saturated branched C₁₋₇ alkyl groups include iso-propyl (C₃), iso-butyl (C₄), sec-butyl (C₄), tert-butyl (C₄), iso-pentyl (C₅), and neo-pentyl (C₅).

C₂₋₇ Alkenyl: The term “C₂₋₇ alkenyl”, as used herein, pertains to an alkyl group having one or more carbon-carbon double bonds. Examples of C₂₋₇ alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₇ Alkynyl: The term “C₂₋₇ alkynyl”, as used herein, pertains to an alkyl group having one or more carbon-carbon triple bonds. Examples of C₂₋₇ alkynyl groups include, but are not limited to, ethynyl (ethinyl, —C≡CH) and 2-propynyl (propargyl, —CH₂—C≡CH).

C₃₋₇ Cycloalkyl: The term “C₃₋₇ cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g., partially unsaturated, fully unsaturated), which moiety has from 3 to 7 carbon atoms. Thus, the term “C₃₋₇ cycloalkyl” includes the sub-classes cycloalkyenyl and cycloalkynyl. Examples of cycloalkyl groups include, but are not limited to, those derived from:
saturated hydrocarbon compounds:
   cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇); and
unsaturated hydrocarbon compounds:
   cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇).

The alkyl groups in the compounds of the invention may optionally be substituted. Substituents include one or more further alkyl groups and/or one or more further substituents, such as, for example, C₅₋₂₀ aryl (e.g. benzyl), C₃₋₂₀ heterocyclyl, amino, cyano (—CN), nitro (—NO₂), hydroxyl (—OH), ester, halo, thiol (—SH), thioether and sulfonate (—S(═O)₂)OR, where R is wherein R is a sulfonate substituent, for example, a C₁₋₇ alkyl group, a C₃₋₂₀ heterocyclyl group, or a C₅₋₂₀ aryl group, preferably a C₁₋₇ alkyl group).

C₃₋₂₀ Heterocyclyl: The term “C₃₋₂₀ heterocyclyl”, as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g., C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₂₀ heterocyclyl, C₅₋₂₀ heterocyclyl, C₅₋₂₀ heteroaryl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and
N₁O₁S₁: oxathiazine (C₆).
C₃₋₂₀ heterocyclyl groups may optionally be substituted with one or more substituents including, for example, C₁₋₇ alkyl, C₅₋₂₀ aryl, C₃₋₂₀ heterocyclyl, amino, cyano, nitro, hydroxyl, ester, halo, thiol, thioether and sulfonate.
C₅₋₂₀ Aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. Preferably, each ring has from 5 to 7 ring atoms.

In this context, the prefixes (e.g., C₃₋₂₀, C₅₋₇, O₅₋₆, etc.) denote the number of ring atoms, or range of number of ring atoms, whether carbon atoms or heteroatoms. For example, the term “C₅₋₆ aryl”, as used herein, pertains to an aryl group having 5 or 6 ring atoms. The ring atoms may be all carbon atoms, as in “carboaryl groups”. Examples of carboaryl groups include C₃₋₂₀ carboaryl, C₅₋₂₀ carboaryl, C₅₋₁₅ carboaryl, C₅₋₁₂ carboaryl, C₅₋₁₀ carboaryl, C₅₋₇ carboaryl, C₅₋₆ carboaryl, C₅ carboaryl, and C₆ carboaryl.

Examples of carboaryl groups include, but are not limited to, those derived from benzene (i.e., phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g., 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of heteroaryl groups include C₃₋₂₀ heteroaryl, C₅₋₂₀ heteroaryl, C₅₋₁₅ heteroaryl, C₅₋₁₂ heteroaryl, C₅₋₁₀ heteroaryl, C₅₋₇ heteroaryl, C₅₋₆ heteroaryl, C₅ heteroaryl, and C₆ heteroaryl.

Examples of monocyclic heteroaryl groups include, but are not limited to, those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heteroaryl groups which comprise fused rings, include, but are not limited to:
C₉ heteroaryl groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀ heteroaryl groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ heteroaryl groups (with 2 fused rings) derived from benzodiazepine (N₂);
C₁₃ heteroaryl groups (with 3 fused rings) derived from carbazole (N₁), dibenzofuran (O₁), dibenzothiophene (S₁), carboline (N₂), perimidine (N₂), pyridoindole (N₂); and,
C₁₄ heteroaryl groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

Tricyclic Heteraromatic Moiety: The term tricyclic heteroaromatic moiety refers to heteroaromatic groups having three fused rings, examples of which are given above with reference to heteroaryl groups.

C₅₋₂₀ aryl groups may optionally be substituted with one or more substituents including, for example, C₁₋₇ alkyl, C₅₋₂₀ aryl, C₃₋₂₀ heterocyclyl, amino, cyano, nitro, hydroxyl, ester, halo, thiol, thioether and sulfonate.

Halo: —F, —Cl, —Br, and —I.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. If R is a C₁₋₇ alkyl group, then the ester may be termed a C₁₋₇ alkyl ester, and if R is a C₅₋₂₀ aryl group, then the ester may be termed a C₅₋₂₀ aryl ester. Examples of ester groups include, but are not limited to, —C(═O)OCH₃, —C(═O)OCH₂ CH₃, —C(═O)OC(CH₃)₃, and —C(═O)OPh.

Amino: —NR¹R², wherein R¹ and R² are independently amino substituents, for example, hydrogen, a C₁₋₇ alkyl group (also referred to as C₁₋₇ alkylamino or di-C₁₋₁₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂ CH₃)₂, —NHCH₂ Ph and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂ CH₃, and —C(═O)N(CH₂ CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂ CH₃ (propionyl), —C(═O)C(CH₃)₃ (t-butyryl), and —C(═O)Ph (benzoyl, phenone).

Sulfo: —S(═O)₂OH, —SO₃H.

Sulfonamido (sulfinamoyl; sulfonic acid amide; sulfonamide): —S(═O)₂ NR¹R², wherein R¹ and R² are independently amino substituents, as defined for amino groups. Examples of sulfonamido groups include, but are not limited to, —S(═O)₂ NH₂, —S(═O)₂ NH(CH₃), —S(═O)₂ N(CH₃)₂, —S(═O)₂ NH(CH₂ CH₃), —S(═O)₂ N(CH₂ CH₃)₂, and —S(═O)₂ NHPh.

Ether: —OR, wherein R is an ether substituent, for example, a C₁₋₇ alkyl group (also referred to as a C₁₋₇ alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂ CH₃.

Azo: —N═N—R, where R is an azo substituent, for example a C₁₋₇ alkyl group, a C₃₋₂₀ heterocyclyl group, or a C₅₋₂₀ aryl group, preferably a C₁₋₇ alkyl group. Examples of azo groups include, but are not limited to, —N═N—CH₃ and —N═N-Ph.

Heterocyclic ring: The term “heterocyclic ring” as used herein refers to a 3-, 4-, 5-, 6-, 7-, or 8-(preferably 5-, 6- or 7-) membered saturated or unsaturated ring, which may be aromatic or non-aromatic, containing from one to three heteroatoms independently selected from N, O and S, e.g. indole (also see above).

Carbocyclic ring: The term “carbocyclic ring” as used herein refers to a saturated or unsaturated ring, which may be aromatic or non-aromatic, containing from 3 to 8 carbon atoms (preferably 5 to 7 carbon atoms) and includes, for example, cyclopropane, cyclobutane, cyclopentane, cyclohexane and cycloheptane (also see above).

### Includes Other Forms

Unless otherwise specified, included in the above are the well known ionic, solvate, and protected forms of these substituents. For example, a reference to carboxylic acid (—COOH) also includes the anionic (carboxylate) form (—COO⁻) or solvate thereof, as well as conventional protected forms. Similarly, a reference to an amino group includes the protonated form (—N⁺HR¹R²) or solvate of the amino group, as well as conventional protected forms of an amino group. Similarly, a reference to a hydroxyl group also includes the anionic form (—O⁻) or solvate thereof, as well as conventional protected forms.

In compounds of formula I, where the ligand is:

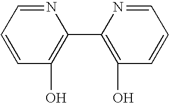

or a substituted version thereof, the hydroxy groups are acidic and one or both may be in their anionic forms (Constable, E. C. and Seddon, K. R., J. Chem. Soc., Chem. Commun., 1982, 34-36) or may be hydrogen bonded (Cargill Thompson, A. M. W., et al., J. Chem. Soc., Dalton Trans., 1996, 879-884).

### Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂ OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

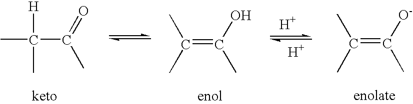

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof, for example, a mixture enriched in one enantiomer. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

Unless otherwise specified, a reference to a particular compound also include solvate forms thereof.

### Chemically Protected Forms

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂ C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀ aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

### Use of Compounds of the Invention

The invention provides compounds of formula (I), or solvates thereof (“active compounds”), for use in a method of treatment of the human or animal body. Such a method may comprise administering to such a subject a therapeutically-effective amount of an active compound, preferably in the form of a pharmaceutical composition.

The term “treatment” as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “therapeutically-effective amount” as used herein, pertains to that amount of an active compound, or a material, composition or dosage form comprising an active compound, which is effective for producing some desired therapeutic effect, commensurate with a reasonable benefit/risk ratio.

### Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

### Formulations

While it is possible for the active compound to be administered alone, it is preferable to present it as a pharmaceutical composition (e.g. formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavoured basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required.

Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

### Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

### Cancers

Examples of cancers which may be treated by the active compounds include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumor of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumor of the central or peripheral nervous system, for example astrocytoma, neuroblastoma, glioma or schwannoma; melanoma; seminoma; teratocarcinoma; osteosarcoma; xenoderoma pigmentoum; keratoctanthoma; thyroid follicular cancer; or Kaposi's sarcoma.

Examples of other therapeutic agents that may be administered together (whether concurrently or at different time intervals) with the compounds of the formula (I) include but are not limited to topoisomerase inhibitors, alkylating agents, antimetabolites, DNA binders and microtubule inhibitors (tubulin target agents), such as cisplatin, cyclophosphamide, doxorubicin, irinotecan, fludarabine, 5FU, taxanes, mitomycin C or radiotherapy. For the case of active compounds combined with other therapies the two or more treatments may be given in individually varying dose schedules and via different routes.

The combination of the agents listed above with a compound of the present invention would be at the discretion of the physician who would select dosages using his common general knowledge and dosing regimens known to a skilled practitioner.

Where the compound of the formula (I) is administered in combination therapy with one, two, three, four or more, preferably one or two, preferably one other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

### Preferences

### R¹—R⁶

In one group of embodiments of the present invention, R¹ and R² together with the ring to which they are attached form a saturated or unsaturated carbocyclic or heterocyclic group containing up to 3- to 8-membered carbocyclic or heterocyclic rings, wherein each carbocyclic or heterocyclic ring may be fused to one or more other carbocyclic or heterocyclic rings.

In this group of embodiments, it is preferred that R³, R⁴, R⁵ and R⁶ are H.

R¹ and R² together with the ring to which they are bound in compounds of formula (I) may represent an ortho- or peri-fused carbocyclic or heterocyclic ring system.

R¹ and R² together with the ring to which they are bound may represent a wholly carbocyclic fused ring system such as a ring system containing 2 or 3 fused carbocyclic rings, e.g. optionally substituted, optionally hydrogenated naphthalene or anthracene.

Alternatively, R¹ and R² together with the ring to which they are bound in compounds of formula (I) may represent a fused tricyclic ring such as anthracene or a mono, di, tri, tetra or higher hydrogenated derivative of anthracene. For example, R¹ and R² together with the ring to which they are bound in formula (I) may represent anthracene, 1,4-dihydroanthracene or 1,4,9,10-tetrahydroanthracene.

R¹ and R² together with the ring to which they are bound in formula (I) may also represent:

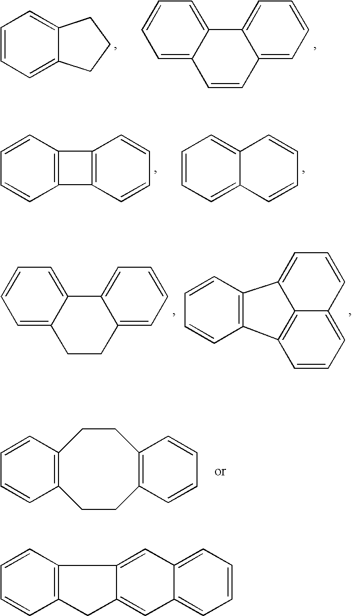

In another group of embodiments, R¹, R², R³, R⁴, R⁵ and R⁶ are independently selected from H, C₁₋₇ alkyl, C₅₋₂₀ aryl, C₃₋₂₀ heterocyclyl, halo, ester, amido, acyl, sulfo, sulfonamido, ether, thioether, azo and amino. In this group of embodiments, R¹, R², R³, R⁴, R⁵ and R⁶ are preferably independently selected from H, C₁₋₇ alkyl, C₅₋₂₀ aryl and ester. Of these H and C₁₋₇ alkyl (in particular C₁₋₃ alkyl) are most preferred.

In this group of embodiments, four, five or six of R¹, R², R³, R⁴, R⁵ and R⁶ are preferably hydrogen, with the other (if any) groups being selected from C₁₋₇ alkyl, C₅₋₂₀ aryl, C₃₋₂₀ heterocyclyl, halo, ester, amido, acyl, sulfo, sulfonamido, ether, thioether, azo and amino, or more preferably C₁₋₇ alkyl, C₅₋₂₀ aryl and ester, and most preferably C₁₋₇ alkyl (in particular C₁₋₃ alkyl). If two of R¹, R², R³, R⁴, R⁵ and R⁶ are not H, then these groups are preferably meta or para to one another, and more preferably para to one another.

Examples of particularly preferred substituent patterns include, but are not limited to: phenyl; 1-methyl; and 4-iso-propyl.

In the group of further embodiments where R¹ is C₅₋₂₀ aryl and R², R³, R⁴, R⁵ and R⁶ are independently selected from H, C₁₋₇ alkyl, C₅₋₂₀ aryl, C₃₋₂₀ heterocyclyl, halo, ester, amido, acyl, sulfo, sulfonamido, ether, thioether, azo and amino, the preferences discussed above may also apply as appropriate. Therefore, in this group of embodiments R², R³, R⁴, R⁵ and R⁶ are preferably independently selected from H, C₁₋₇ alkyl, C₅₋₂₀ aryl and ester. Of these H and C₁₋₇ alkyl (in particular C₁₋₃ alkyl) are most preferred.

In this group of further embodiments, four or five of R², R³, R⁴, R⁵ and R⁶ are preferably hydrogen, with the other (if any) groups being selected from C₁₋₇ alkyl, C₅₋₂₀ aryl, C₃₋₂₀ heterocyclyl, halo, ester, amido, acyl, sulfo, sulfonamido, ether, thioether, azo and amino, or more preferably C₁₋₇ alkyl, C₅₋₂₀ aryl and ester, and most preferably C₁₋₇ alkyl (in particular C₁₋₃ alkyl).

Preferred arene groups in this group of further embodiments include dibenzene and terpene.

### X

X is preferably halo and is more preferably I or Cl.

### R^{N1}, R^{N2}

In one embodiment, R^{N1} and R^{N2} are preferably independently selected from hydroxyl, C₁₋₇ (more preferably C₁₋₄) alkoxy, carboxy and C₁₋₇ (more preferably C₁₋₄) alkyl ester. More preferably they are independently selected from hydroxyl, methoxy, carboxy and methyl ester, of which hydroxy are methoxy are most preferred.

In some embodiments, R^{N1} and R^{N2} are the same, for example hydroxy.

In another embodiment, R^{N1}, R^{N2} together with the pyridine rings to which they are attached, form a tricyclic heteroaromatic moiety where the third ring also has six ring atoms. A particularly preferred group is:

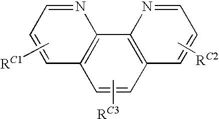

### R^{C1}, R^{C2}, R^{C3} (If Present)

R^{C1}, R^{C2} and R^{C3} (if present) are preferably independently selected from hydroxyl, C₁₋₇ (more preferably C₁₋₄) alkoxy, carboxy and C₁₋₇ (more preferably C₁₋₄) alkyl ester. More preferably they are independently selected from hydroxyl, methoxy, carboxy and methyl ester, of which hydroxy are methoxy are most preferred.

In some embodiments, R^{C1} and R^{C2} are the same, for example hydroxy.

If one of both R^{C1} and R^{C2} are present, they are both preferably para to the N ring atom.

In some embodiments, it is preferred that R^{C1}, R^{C2} and R^{C3} (if applicable) are not present, i.e. the ligand is unsubstituted, except for R^{N1} and R^{N2}.

There may be one, two or three of R^{C1} and R^{C2}, and one or two of R^{C3} (if present).

### Y^{q}

Y^{q} in compounds of formula (I) is a counterion and is only present in the compound when the complex containing the metal ion is charged. If m is +1 or +2, then Y^{q} is preferably a non-nucleophilic anion such as PF₆⁻, BF₄⁻, BPh₄⁻ or CF₃O₂SO⁻, for example. If m is −1, then Y^{q} is preferably a cation such as NH₄⁺, K⁺, Na⁺, Cs⁺. Imidazolium and indazolium cations may also be used.

### General Synthesis Methods

The present invention also provides a process for preparing the compounds of the invention which comprises the reaction of a dimeric ruthenium complex of formula [(η⁶-C₆ (R¹)(R²)(R³)(R⁴)(R⁵)(R⁶))RuX₂]₂ with an appropriate ligand in the presence, or with subsequent addition of, Y^{q} (if necessary), in a suitable solvent for the reaction, wherein R¹, R², R³, R⁴, R⁵, R⁶, X, and Y are as defined above for the compounds of the invention.

Preferred reaction conditions include:
(a) stirring the starting dimeric ruthenium complex, as described above, in MeOH or a MeOH/water mixture;
(b) adding the ligand as a solution in MeOH;
(c) stirring the resultant solution at room temperature; and
(d) adding a source of Y^{q} (if necessary), such as a compound of formula (NH₄⁺)Y^{q}, if q is negative, e.g., NH₄PF₆, or Y^{q}Cl, if q is positive, e.g., KCl, and filtering off the precipitated product.

The following non-limiting examples illustrate the present invention.

### EXAMPLES

### General Methods

Materials: The starting materials [(η⁶-arene)RuCl₂]₂ (arene=indan, tetrahydroanthracene (THA), dihydroanthracene (DHA), benzene (bz), biphenyl (biph), p-terphenyl (p-terp)) were prepared according to the literature (Chen, H., et al., J. Am. Chem. Soc., 2002, 124, 3064-3082; Wang, F., et al., Proc. Natl. Acad. Sci. U.S.A., 2005, 102, 18269-18274). The following materials were used in the examples: RuCl₃.xH₂O was purchased from Alfa-Aesar. Indan, hexamethylbenzene, fluorene, phenanthroline, bipy, 2,9-Me₂-phenanthroline, 4,4′-Me-bipy, 3,3′-dihydroxy-2,2′-bipyridine were obtained from Aldrich. Fluorene and diaminophenylene were further purified by sublimation. 4,4′-(CO₂ Me)-bipy and 4,4′ (CH₂OH)-bipy were prepared according a published procedure (Wiederholt, K. and McLaughlin, L. W., Nucleic Acids Research, 1999, 27, 2487-2493). Acetonitrile was dried over CaH₂, alcohols were dried and distilled from Mg/I₂. THF were dried over Na/benzophenone. Diethylether, and hexane was distilled over Na metal prior to use. All other reagents were obtained from commercial suppliers and used as received.
NMR-Spectroscopy: All ¹H NMR experiments for characterisation of synthesised compounds were recorded on either a Bruker DMX 500 MHz spectrometer equipped with TBI [¹H, ¹³C, ¹⁵ N] probe-head, equipped with z-field gradients or a Bruker DPX 360 MHz spectrometer. The proton signals were calibrated against the residual solvent peak, δ 7.27 (chloroform), 2.07 (acetone) and 2.52 (DMSO). The 2D ¹H-TOCSY and 2D ¹H COSY experiments for characterisation were run on a Bruker DMX 500 MHz spectrometer. 2D-¹H ROESY experiment for characterisation was recorded on a Bruker AVA 600 mHz spectrometer equipped with a with a TXI [¹H, ¹³C, ¹⁵ N] probe-head, equipped with z-field gradients. All pH titration experiments were recorded on a Bruker AVA 600 MHz spectrometer where dioxan was added as an internal reference (δ 3.75, in 100% D₂O). The water was suppressed using a 1D Double Pulse Field Gradient Spin Echo (DPFGSE) experiment. The aqueous solution behaviour was recorded on a Bruker bio 600 MHz spectrometer equipped with a cryoprobe and the water was suppressed using a 1D Double Pulse Field Gradient Spin Echo (DPFGSE) experiment. The chemical shifts were measured relative to dioxin (internal reference δ 3.75, in 90% H₂O/10% D₂O). All spectra were recorded using 5 mm quartz tubes at 298 K unless stated otherwise. All NMR data were processed using Xwin-NMR (Version 2.0 Bruker UK Ltd).
Elemental Analysis: Elemental analysis was carried out by the University of Edinburgh using an Exeter analytical elemental analyser CE440.
Electrospray Mass Spectrometry: ESI-MS were obtained on a Micromass Platform II Mass Spectrometer and solutions were infused directly. The capillary voltage was 3.5 V and the cone voltage used was dependent on the solution (typically varied between 5-15 V). The source temperature was ca. 383 K.

### Example 1

### [(η⁶-indan)RuCl(bipydiol-N,N)Cl](1)

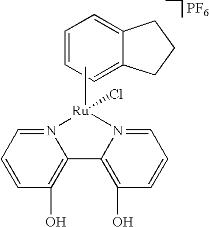

The dimer [Ru(indan)Cl₂]₂ (0.075 g, 0.13 mmole) was suspended in MeOH (10 mL), and to this bipyridine-diol (0.050 g, 0.26 mmole) in MeOH (3 mL) was added drop wise. The reaction mixture was left stirring at ambient temperature for 1 hour. It was then filtered and to the filtrate NH₄PF₆ (0.128 g, 0.80 mmole) was added and the flask shaken. Precipitate started to appear almost immediately. The flask was kept at 253K overnight. The solid obtained was collected by filtration, washed with cold methanol and ether and dried in air to give an intense yellow solid.

Yield, 52%; ¹H NMR (DMSO-d₆): δ 8.60 (m, 2H), 7.27 (m, 2H), 7.05 (m, 2H), 6.10 (m, 2H) 5.82 (m, 2H), 2.67-250, 2.05-1.85 (m, 6H), Anal. Calcd for C₁₉H₁₈ ClF₆N₂O₂ PRu: C, 39.73; H, 3.67; N, 4.63. Found: C, 39.03; H, 3.66; N, 4.66.

### Example 2

### [(η⁶-indan)Ru(phenanthroline-N,N)Cl]PF₆ (2)

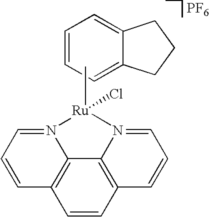

The dimer [Ru(indan)Cl₂]₂ (0.150 g, 0.26 mmole) was suspended in MeOH (40 mL), and to this phenanthroline (0.103 g, 0.52 mmol) in MeOH (10 mL) was added dropwise. The resulting clear solution turned golden-yellow. The reaction mixture was left stirring at ambient temperature for 18 hours. It was then filtered and the volume of the filtrate was reduced on a rotary evaporator to ca. 15 mL at which point NH₄PF₆ (0.127 g, 0.78 mmol) was added and the flask shaken. A yellowish precipitate started to appear almost immediately. The flask was kept at 253 K for 1 hour. The solid obtained was collected by filtration, washed with cold methanol and ether and dried in air.

Yield: 260 mg, 85%. Crystals suitable for X-ray analysis were obtained buy slow evaporation of a methanolic solution at ambient temperature. ¹H NMR (DMSO-d₆): δ 9.85 (m, 2H), 8.9 (m, 2H), 8.26 (s, 2H), 8.14 (m, 2H) 6.47 (m, 2H), 6.02 (m, 2H), 2.72 (m, 4H) 1.86 (m, 1H), 1.41 (m, 1H). Anal. Calcd for C₂₁H₂₂ ClF₆N₂PRu: C, 43.44; H, 3.80; N, 4.82. Found: C, 43.41; H, 3.81; N, 4.68.

### Example 3

### [(η-indan)Ru(2,9-Me₂-phen-N,N)Cl]PF₆ (3)

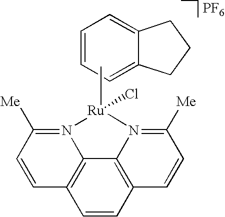

The dimer [Ru(indan)Cl₂]₂ (0.122 g, 0.21 mmole) was suspended in MeOH (20 mL) and to this neocuprione (2,9-Me₂-phenanthroline) (0.088 g, 0.42 mmol) was added dropwise and the reaction mixture stirred for 3 hours. It was then filtered and the volume of the filtrate was reduce on a rotary evaporator to ca 7 mL at which point NH₄PF₆ (0.108 g, 0.62 mmol) was added and the flask kept at 253 K overnight. A yellowish crystalline solid were collected by filtration, washed with cold methanol and ether and dried in air.

Yield 90 mg, 35%. ¹H NMR (DMSO-d₆): δ 8.74 (m, 2H), 8.16 (m, 2H), 8.07 (m, 2H), 6.43 (m, 2H) 6.13 (m, 2H) 3.11 (m, 6H), 1.92 (m, 2H), 1.58 (m, 2H), 0.87 (m, 1H), 0.35 (m, 1H). Anal. Calcd for C₂₃H₂₅ ClF₆N₂PRu: C, 45.22; H, 3.64; N, 4.60. Found: C, 45.21; H, 3.79; N, 4.55

### Example 4

### [(η⁶-THA)Ru(bipydiol-N,N—H)Cl](4)

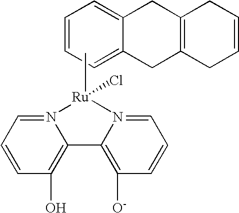

[(η⁶-THA)RuCl₂]₂ (0.05 g, 0.07 mmole) was suspended in dry, freshly distilled methanol (25 ml). Into this suspension 2,2′-bypiridine-3,3′-diol (0.026 g, 0.14 mmol) was added. The reaction mixture was stirred at room temperature in argon atmosphere overnight. The resulting clear yellow solution was filtered. NH₄PF₆ (0.1 g, 0.6125 mmol) was added into this. The volume was reduced until precipitation was observed. It was kept at 277 K for 24 hours for further precipitation. The fine yellow precipitate was collected by filtration, washed with a little methanol followed by ether, and dried in vacuum. It was recrystallized from methanol/ether

Yield: 0.037 g, 51%; ¹H NMR (DMSO-d₆): δ 17.92 (s, 1H, OH), ²⁶ 8.62 (d, 2H), 7.22 (t, 2H), 7.06 (d, 2H), 6.08 (d of d, 2H), 5.91 (d of d, 2H), 5.56 (s, 2H), 3.12 (m, 2H), 2.43 (m, 4H), 1.92 (m, 2H). Anal. Calculated for C₂₄H₂₁ ClN₂O₂Ru: C, 56.97; H, 4.18; N, 5.54. Found: C, 50.48; H, 3.73; N, 5.11.

Compound 4 was shown to react with ethylguanine, and from X-ray studies, the apparent arene-bipyridyl stacking, rather than stacking between the arene and the purine ring of guanine was seen.

### Example 5

### [(η⁶-DHA)Ru(bipydiol-N,N—H)Cl](5)

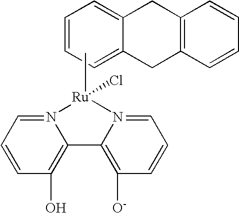

[(η⁶-DHA)RuCl₂]₂ (0.028 g, 0.04 mmol) was suspended in dry, freshly distilled methanol (30 ml). Into this suspension 2,2′-bypyridine-3,3′-diol (0.015 g, 0.08 mmol) was added. The reaction mixture was stirring at room temperature in argon atmosphere overnight. The resulting clear yellow solution was filtered. NH₄PF₆ (0.033 g, 0.2 mmol) was added into this. The volume was reduced until precipitation was observed. It was kept at 277 K for 24 hours for further precipitation. The fine yellow precipitate was collected by filtration, washed with a little methanol followed by ether, and dried in vacuum. It was recrystallized from methanol/ether.

Yield 0.021 g, 41%; ESI-MS: m/z 504.8; ¹H NMR (DMSO-d₆): 17.69 (s, 1H, OH), 8.62 (d, 2H), 7.14 (t, 2H), 6.95 (d, 2H), 6.89 (d, 2H), 6.63 (t, 2H), 6.30 (d, 2H), 5.96 (t, 2H), 4.17 (d, 2H), 3.77 (d, 2H). Anal. Calculated for C₂₄H₁₉ ClN₂O₂Ru: C, 57.20; H, 3.80; N, 5.56. Found: C, 51.44; H, 2.97; N, 5.04.

### Example 6

### [(η⁶-THN)Ru(bipydiol-N,N—H)Cl](6)

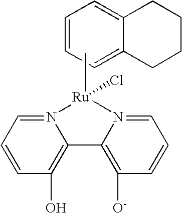

[(η⁶-THN)RuCl₂]₂ (0.03 g, 0.05 mmole) was suspended in dry, freshly distilled methanol (30 ml). Into this suspension 2,2′-bypyridine-3,3′-diol (0.018 g, 0.10 mmol) was added. The reaction mixture was stirring at room temperature in argon atmosphere for 4 hours. The resulting clear yellow solution was filtered. NH₄PF₆ (0.02 g, 0.15 mmol) was added into this. The volume was reduced until precipitation was observed. It was kept at 277 K for 24 hours for further precipitation. The fine yellow precipitate was collected by filtration, washed with a little methanol followed by ether, and dried in vacuum. It was recrystallized from methanol/ether.

Yield: 0.040 g, 68%; ESI-MS: m/z 457.3; Anal. Calculated for C₂₀H₂₀RuClN₂PF₆: C, 46.60; H, 3.32; N, 5.29. Found: C, 39.9; H, 3.35; N, 4.65; ¹H NMR (DMSO-d₆): 17.92 (s, 1H, OH), 8.66 (d, 2H), 7.27 (t, 2H), 7.10 (d, 2H), 5.96 (t, 2H), 5.84 (d, 2H), 1-3 (m, 8H). Anal. Calculated for C₂₀H₁₉ RuClO₂N₂: C, 52.69; H, 4.20; N, 6.14. Found: C, 47.52; H, 3.92; N, 5.34.

### Example 7

### [(η⁶-bz)RuCl(bipy(OH)O⁻)](7)

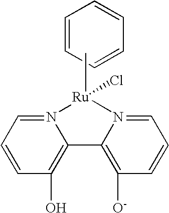

[(η⁶-bz)RuCl₂]₂ (0.052 g, 0.106 mmole) was suspended in dry, freshly distilled methanol (30 ml). Into this suspension 2,2′-bipyridine-3,3′-diol (0.040 g, 0.213 mmol) was added. The reaction mixture was stirred at room temperature in argon atmosphere for 3 hours. The precipitation was observed. It was kept at 277 K for 24 h for further precipitation. The fine yellow precipitate was collected by filtration, washed with a little methanol followed by ether, and dried in vacuum. It was recrystallized from methanol/ether.

Yield: 72.0%. ¹H NMR in DMSO-d₆: δ 17.89 (s, 1H, OH), 8.78 (d, 2H), 7.21 (d of d, 2H), 7.09 (d, 2H), 6.05 (s, 6H). Anal. Calculated for C₁₆H₁₃ ClN₂O₂Ru: C, 47.83; H, 3.26; N, 6.97. Found: C, 46.72; H, 2.83; N, 6.68.

### Example 8

### [(η⁶-biph)RuCl(bipy(OH)O⁻)](8)

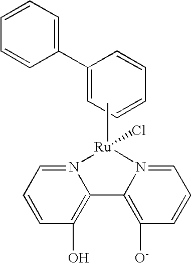

[(η⁶-biph)RuCl₂]₂ (0.053 g, 0.08 mmole) was suspended in dry, freshly distilled methanol (30 ml). Into this suspension 2,2′-bipyridine-3,3′-diol (0.030 g, 0.16 mmol) was added. The reaction mixture was stirred at room temperature in argon atmosphere overnight. The volume was reduced until precipitation was observed. The fine yellow precipitate was collected by filtration, washed with a little methanol followed by ether, and dried in vacuum. It was recrystallized from acetone/ether.

Yield: 63.0%. ¹H NMR in DMSO-d₆: δ 17.85 (s, 1H, OH), 8.5 (d, 2H), 7.7 (t, 1H), 7.6 (d, 2H), 7.4 (t, 2H), 7.00 (m, 4H), 6.5 (d, 2H), 6.2 (t, 2H), 6.1 (t, 1H). Anal. Calculated for C₂₂H₁₇ ClN₂O₂Ru: C, 55.29; H, 3.58; N, 5.86. Found: C, 55.36; H, 3.27; N, 5.88.

### Example 9

### [(η⁶-p-terp)RuCl(bipy(OH)O—)](9)

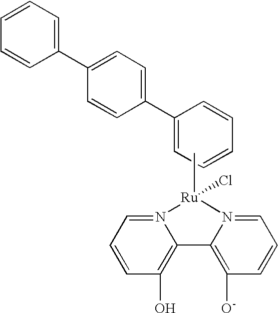

[(η⁶-p-terph)RuCl₂]₂ (0.050 g, 0.062 mmole) was suspended in dry, freshly distilled methanol (50 ml). Into this suspension 2,2′-bipyridine-3,3′-diol (0.023 g, 0.124 mmol) was added. The reaction mixture was stirred at room temperature in argon atmosphere overnight. The volume was reduced until precipitation was observed. The fine yellow precipitate was collected by filtration, washed with a little methanol followed by ether, and dried in vacuum. It was recrystallized from acetone/ether. Yield: 74.6%. ¹H NMR in DMSO-d₆: δ 17.91 (s, 1H, OH), 8.5 (d, 2H), 7.75 (m, 6H), 7.5 (t, 2H), 7.4 (t, 1H), 7.1 (m, 4H), 6.5 (d, 2H), 6.2 (t, 2H), 6.1 (t, 1H). Anal. Calculated for C₂₈H₂₁ ClN₂O₂Ru: C, 60.70; H, 3.80; N, 5.06. Found: C, 59.22; H, 3.41; N, 4.69.

### Example 10

### Cytotoxicity Studies

Some compounds were tested for cytotoxicity against the A2780 ovarian cancer cell line as follows.

The human ovarian cells were added at a density of 1×10⁴ cells per well to 24-well tissue culture trays (Falcon Plastic, Becton Dickenson, Lincon Park, N.J., USA) and allowed to grow for 72 hours before addition of the Ru(II) arene complexes. Stock solutions of the ruthenium compounds were made up fresh in deionised water and sonicated to ensure complete dissolution. These stock solutions were diluted with media to give final concentrations ranging between 0.1 and 100 μM. All compounds were evaluated at each concentration in duplicate wells, and complete assays were repeated a minimum of three times. Cisplatin or carboplatin was employed as a positive and comparative control in each experiment. After 24 hours exposure the drug-containing medium was removed, the cells washed with phosphate buffered saline (PBS) and fresh medium was added. Cell number was assessed after a further 72 h growth using s Coulter counter (Coulter Electronics Ltd, Luton, UK) and the IC₅₀ values (concentration of drug causing 50% growth inhibition) calculated by linear regression analysis comparing the inhibitory effects of the drugs against the growth of untreated cells.

| Compound | IC₅₀ (μM) |
|---|---|
| 1 | 3 |
| 2 | 52 |
| 3 | >50 |

Compound 1 was also tested against the cisplatin resistant cell line (A2780^{cis}) and showed an IC₅₀ of 2.1 μM, i.e. a 0.7 fold resistance.

### Example 11

### Further Cytotoxicity Studies

Other compounds were tested for inhibitory growth activity against the A2780 and A549 cancer cell lines as follows. Each drug was tested for activity at six different concentrations (100 μM, 50 μM, 10 μM, 5 μM, 1 μM and 0.1 μM) and each concentration was tested in triplicate, relative to a cisplatin control.

The A2780 cancer cell line was maintained by growing the cells in RPMI media supplemented with 5% fetal bovine serum, 1% penicillin/streptomycin and 2 mM L-glutamine. The cells were split when approximately 70-80% confluence were reached using 0.25% trypsin/EDTA. The cells were kept incubated at 37° C., 5% CO₂, high humidity. The A549 cancer cell line was maintained by growing the cells in DMEM media supplemented with 10% fetal bovine serum, 1% penicillin/streptomycin and 2 mM L-glutamine. The cells were split when approximately 70-80% confluence were reached using 0.25% trypsin/EDTA. The cells were kept incubated at 37° C., 5% CO₂, high humidity.

A2780 cancer cells were plated out at 5000 cells/well (±10%) on day one. A549 cancer cells were plated out at 2000 cells/well (±10%) on day two. On day three the test compound was dissolved in DMSO to give a stock solution of 20 mM and serial dilutions were carried out in DMSO to give concentrations of drug in DMSO of 10 mM, 2 mM, 1 mM, 0.2 mM and 0.02 mM. These were added to the wells to give the six testing concentrations and a final concentration of DMSO as 0.5% (v/v) with a total volume of drugs and media to be 200 μl. The cells were exposed to the drug for 24 hours then, after drug removal, fresh media was given and the cells were incubated for 96 hours recovery time. The remaining biomass was estimated by the sulforhodamine B assay. The cells were then fixed using 50 μl 50% (w/v) TCA and incubated at 4° C. for one hour. The biomass was stained with 100 μl 0.4% (w/v) sulforhodamine B in 1% acetic acid.

The dye was solubilised with Tris Buffer and the absorbance was read using a BMG Fluostar microplate reader at 595 nm. A baseline correction at 690 nm was subtracted from the values. The absorbance for 100% cell survival was based on the average absorbance for the 0.1 μM dosed triplicate for that drug. IC₅₀ values were calculated using XL-Fit version 4.0.

| Compound | A2780 IC₅₀ (μM) | A549 IC₅₀ (μM) |
|---|---|---|
| 1 | 17.6 | 39 |
| 4 | 7.9 | 21 |
| 5 | 16.7 | 38.4 |
| 6 | 7.3 | 23.6 |
| 7 | 65 | — |
| 8 | 40 | — |
| 9 | 21 | 62 |

1. A pharmaceutical composition comprising a ruthenium (II) compound of formula (I): 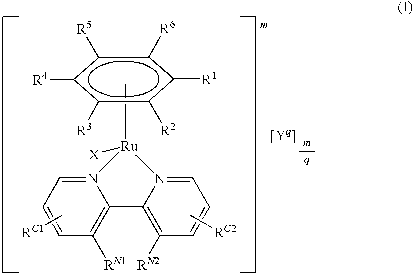
and a pharmaceutically acceptable carrier or diluent wherein:
R¹, R², R³, R⁴, R⁵ and R⁶ are independently selected from H, C₁₋₇ alkyl, C₅₋₂₀ aryl, C₃₋₂₀ heterocyclyl, halo, ester, amido, acyl, sulfo, sulfonamido, ether, thioether, azo and amino, or R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other carbocyclic or heterocyclic rings;
X is halo or a neutral or negatively charged O, N— or S— donor ligand;
Y is a counterion;
m is −1, 0, 1 or 2;
q is 1, 2 or 3;
R^{C1} and R^{C2} independently represent one or more optional substituents selected from hydroxy, C₁₋₇ alkoxy, C₅₋₂₀ aryloxy, C₁₋₇ alkyl, carboxy, C₁₋₇ alkyl ester and C₅₋₂₀ aryl ester;
R^{N1} and R^{N2} are independently selected from hydroxy, C₁₋₇ alkoxy, C₅₋₂₀ aryloxy, C₁₋₇ alkyl, carboxy, C₁₋₇ alkyl ester and C₅₋₂₀ aryl ester;
or R^{N1} and R^{N2} together with the pyridine rings to which they are bound form an tricyclic heteraromatic moiety, where the ring formed by R^{N1} and R^{N2} together may be optionally substituted by one or more substituents represented by R^{C3} selected from: hydroxy, C₁₋₇ alkoxy, C₅₋₂₀ aryloxy, C₁₋₇ alkyl, carboxy, C₁₋₇ alkyl ester and C₅₋₂₀ aryl ester.

2. The composition according to claim 1, wherein R^{N1} and R^{N2} are independently selected from hydroxy, methoxy, carboxy and methyl ester.

3. The composition according to claim 2, wherein R^{N1} and R^{N2} are hydroxy.

4. The composition according to claim 1, wherein R^{N1} and R^{N2}, together with the pyridine rings to which they are attached, form a group which is:

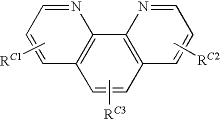

5. The composition according to claim 1, wherein R^{C1} and R^{C2} are independently selected from hydroxy, methoxy, carboxy and methyl ester.

6. The composition according to claim 4, wherein R^{C1}, R^{C2} and R^{C3} are not present.

7. The composition according to claim 1, wherein X is halo.

8. The composition according to claim 7, wherein X is chloro or iodo.

9. The composition according claim 1, wherein R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other carbocyclic or heterocyclic rings.

10. The composition according to claim 9, wherein R³, R⁴, R⁵ and R⁶ are H.

11. The composition according to claim 1, wherein R¹, R², R³, R⁴, R⁵ and R⁶ are independently selected from C₁₋₇ alkyl, C₅₋₂₀ aryl, C₃₋₂₀ heterocyclyl, halo, ester, amido, acyl, sulfo, sulfonamido, ether, thioether, azo and amino.

12. The composition according to claim 11, wherein R¹, R², R³, R⁴, R⁵ and R⁶ are independently selected from H and C₁₋₇ alkyl.

13. The composition according to claim 11, wherein at least four of R¹, R², R³, R⁴, R⁵ and R⁶ are hydrogen.

14. (canceled)

15. (canceled)

16. (canceled)

17. A method of treatment of a subject suffering from cancer, comprising administering to such a subject a therapeutically-effective amount of the pharmaceutical composition of claims 1.

18. A ruthenium (II) compound of formula (I): 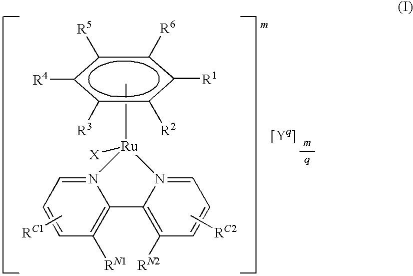
, wherein:
R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other carbocyclic or heterocyclic rings; or
R¹ is C₅₋₂₀ aryl, and R² is selected from H, C₁₋₇ alkyl, C₅₋₂₀ aryl, C₃₋₂₀ heterocyclyl, halo, ester, amido, acyl, sulfo, sulfonamido, ether, thioether, azo and amino;
R³, R⁴, R⁵ and R⁶ are independently selected from H, C₁₋₇ alkyl, C₅₋₂₀ aryl, C₃₋₂₀ heterocyclyl, halo, ester, amido, acyl, sulfo, sulfonamido, ether, thioether, azo, amino, or
X is halo or a neutral or negatively charged O, N— or S— donor ligand;
Y is a counterion;
m is −1, 0, 1 or 2;
q is 1, 2 or 3;
R^{C1} and R^{C2} independently represent one or more optional substituents selected from hydroxy, C₁₋₇ alkoxy, C₅₋₂₀ aryloxy, C₁₋₇ alkyl, carboxy, C₁₋₇ alkyl ester and C₅₋₂₀ aryl ester;
R^{N1} and R^{N2} are independently selected from hydroxy, C₁₋₇ alkoxy, C₅₋₂₀ aryloxy, C₁₋₇ alkyl, carboxy, C₁₋₇ alkyl ester and C₅₋₂₀ aryl ester;
or R^{N1} and R^{N2} together with the pyridine rings to which they are bound form an tricyclic heteraromatic moiety, where the ring formed by R^{N1} and R^{N2} together may be optionally substituted by one or more substituents represented by R^{C3} selected from: hydroxy, C₁₋₇ alkoxy, C₅₋₂₀ aryloxy, C₁₋₇ alkyl, carboxy, C₁₋₇ alkyl ester and C₅₋₂₀ aryl ester.

19. The compound according to claim 18, wherein R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other carbocyclic or heterocyclic rings, and R³, R⁴, R⁵ and R⁶ are H.

20. The compound according to claim 18, wherein R¹ is C₅₋₂₀ aryl and R², R³, R⁴, R⁵ and R⁶ are H.

21. The compound according to claim 18, wherein R¹ is C₅₋₂₀ aryl and R^{N1} and R^{N2} are independently selected from hydroxy, C₁₋₇ alkoxy, C₅₋₂₀ aryloxy, C₁₋₇ alkyl, carboxy, C₁₋₇ alkyl ester and C₅₋₂₀ aryl ester.

22. The compound according to claim 18, wherein R^{N1} and R^{N2} are independently selected from hydroxy, methoxy, carboxy and methyl ester.

23. The compound according to claim 22, wherein R^{N1} and R^{N2} are hydroxy.

24. The compound according to claim 18, wherein R^{N1} and R^{N2}, together with the pyridine rings to which they are attached, form a group which is:

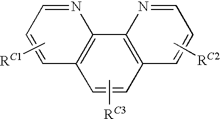

25. The compound according to claim 18, wherein R^{C1} and R^{C2} are independently selected from hydroxy, methoxy, carboxy and methyl ester.

26. The compound according to claim 24, wherein R^{C1}, R^{C2} and R^{C3} are not present.

27. The compound according to any one claim 18, wherein X is halo.

28. The compound according to claim 27, wherein X is chloro or iodo.

29. A ruthenium (II) compound of formula (I): 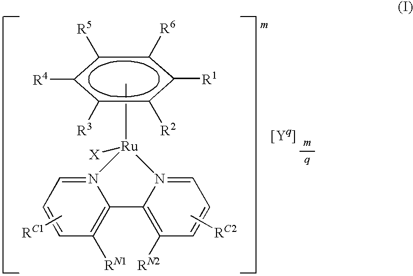
wherein:
R¹, R², R³, R⁴, R⁵ and R⁶ are independently selected from H, C₁₋₇ alkyl, C₃₋₂₀ heterocyclyl, halo, ester, amido, acyl, sulfo, sulfonamido, ether, thioether, azo and amino;
X is halo or a neutral or negatively charged O, N— or S— donor ligand;
Y is a counterion;
m is −1, 0, 1 or 2;
q is 1, 2 or 3;
R^{C1} and R^{C2} independently represent one or more optional substituents selected from hydroxy, C₁₋₇ alkoxy, C₅₋₂₀ aryloxy, C₁₋₇alkyl, carboxy, C₁₋₇ alkyl ester and C₅₋₂₀ aryl ester;
R^{N1} and R^{N2} are independently selected from hydroxy, C₁₋₇ alkoxy, C₅₋₂₀ aryloxy, C₁₋₇ alkyl, carboxy, C₁₋₇ alkyl ester and C₅₋₂₀ aryl ester.

30. The compound according to claim 29, wherein R^{N1} and R^{N2} are independently selected from hydroxy, methoxy, carboxy and methyl ester.

31. The compound according to claim 30, wherein R^{N1} and R^{N2} are hydroxy.

32. The compound according to claim 29, wherein R^{C1} and R^{C2} are independently selected from hydroxy, methoxy, carboxy and methyl ester.

33. The compound according to claim 29, wherein X is halo.

34. The compound according to claim 33, wherein X is chloro or iodo.

35. The compound according to claim 29, wherein R¹, R², R³, R⁴, R⁵ and R⁶ are independently selected from H and C₁₋₇ alkyl.

36. The compound according to claim 35, wherein at least four of R¹, R², R³, R⁴, R⁵ and R⁶ are hydrogen.
